(19) United States
(12) Patent Application Publication
(10) Pub. No.: US 2020/0062598 A1
(43) Pub. Date: Feb. 27, 2020

(54) PLATINUM AND TIN OXIDE CO-FUNCTIONALIZED SINGLED WALLED CARBON NANOTUBES (PT/SNO2/SWNTS) AND THEIR SENSING PROPERTIES TOWARDS CARBON MONOXIDE AT ROOM TEMPERATURE

(71) Applicant: THE REGENTS OF THE UNIVERSITY OF CALIFORNIA, Oakland, CA (US)

(72) Inventors: Nosang Vincent Myung, Riverside, CA (US); Heng C. Su, Riverside, CA (US)

(73) Assignee: THE REGENTS OF THE UNIVERSITY OF CALIFORNIA, Oakland, CA (US)

(21) Appl. No.: 16/344,110

(22) PCT Filed: Oct. 24, 2017
(86) PCT No.: PCT/US2017/058098

§ 371 Date: Apr. 23, 2019

Related U.S. Application Data
(60) Provisional application No. 62/412,120, filed on Oct. 24, 2016.

(51) Int. Cl. C01B 32/174; C01B 32/159; G01N 27/407; C25D 9/04; C25D 3/50; G01N 27/30; G01N 33/00; G01N 27/12

(52) U.S. Cl. CPC C01B 32/174; C01B 32/159; G01N 27/4075; C25D 9/04; C25D 3/50; C01B 2202/22; G01N 33/004; G01N 27/127; C01B 2202/02; C01B 2202/08; C01P 2004/03; G01N 27/308

(57) ABSTRACT

A method and system are disclosed of Pt and SnO₂ co-functionalized on single-walled carbon nanotubes (SWNTs) assembled on microelectrodes by electrochemical deposition where Pt nanoparticle's morphology, size, and density were tuned by controlling electrodeposition potential and time. The method and system to obtain the optimum condition for Pt decorated SnO₂/SWNTs (Pt/SnO₂/SWNTs) were performed and also correlate with its CO sensing performance. Light dependent sensing performance was examined with red, green and UV LED light under room temperature. With the assistance of the UV LED light illumination, the sensitivity of Pt/SnO₂/SWNTs was further enhanced to 2.1%/ppm_{V} to 50 ppm_{V} of CO and the detection limit can push down to 0.05 ppm_{V}.

### CROSS-REFERENCES TO RELATED APPLICATIONS

This application claims priority to U.S. Provisional Application No. 62/412,120 filed on Oct. 24, 2016, the entire content of which is incorporated herein by reference.

### FIELD OF THE INVENTION

The present disclosure relates to platinum (Pt) and tin oxide (SnO₂) co-functionalized on single-walled carbon nanotubes (SWNTs) assembled on microelectrodes by electrochemical deposition where Pt nanoparticle's morphology, size, and density were tuned by controlling electrodeposition potential and time.

### BACKGROUND OF THE INVENTION

Carbon monoxide (CO) is a common by-product generated by incomplete combustion of carbon-containing materials. Common sources of CO can include heating units, gas stoves, automobile exhaust gases, etc. Carbon monoxide intoxication is one of the most common types of fatal poisoning throughout the world including the United States. For example, CO can cause severe damage to the heart and brain when the carboxyhemoglobin (COHb) level in the blood exceeds 20%. This is because the affinity of hemoglobin for CO is 210 times higher than that for O₂ resulting in hypoxia which may cause headache, nausea, loss of conscious or even death. Moreover, CO becomes unstable and can become explosive when its volume percent concentration ranges between 12.5% and 74.2% in air. In addition, CO's intrinsic properties of being colorless, odorless, tasteless and initially non-irritating make it very difficult to detect by conventional methods. Thus, it is crucial to develop new types of sensors that can detect CO below 50 ppm_{V} (personnel exposure limit for CO) levels in the environment with high sensitive and rapid response.

The catalytic oxidation of CO to CO₂ has long been a benchmark reaction in heterogeneous catalysts especially in many industrial processes, including the reduction of CO in automobile exhaust gases and the selective reduction in fuel cells. During the past decades, considerable efforts have been devoted to CO oxidation by noble metals in the form of single crystal or as nanoparticles in the supported metal oxides. The influence of the metal particle sizes and density (on the support) have been studied due to its significance from both fundamental and practical view of points. The activity of the catalytic oxidation with varying metal particle size (for example, 1 nm to 10 nm) and density is corresponding to the available active sites, electronic states, interaction of metal and support, and oxidation states. The interaction of surface adsorbed oxygen and the CO molecules is believed to be the main step in the oxidation process. There are two popular mechanisms to describe the entire oxidation process, the Langmuir-Hinselwood mechanism (L-H) and the Eley-Rideal mechanism (E-R). In the L-H mechanism, a CO molecule in the gas phase is first adsorbed onto the metal surface. Then it reacts with a surface adsorbed oxygen molecule to form the product, CO₂, which is released to the air under the assumption of an isothermal steady state condition. In general, the CO oxidation on the Pt surface can for the L-H mechanism be simplified as shown in equations 1-3.

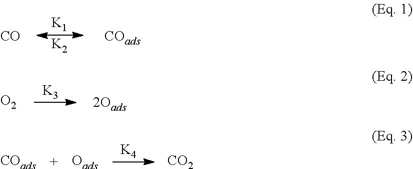

In the E-R mechanism, a CO molecule in the gas phase reacts directly with a previously adsorbed oxygen molecule, without being adsorbed itself, to release the product CO₂ into the air. For the E-R mechanism, the oxidation process can be expressed as following in equations 4 and 5.

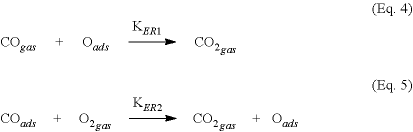

The resulting rate law is typically complicated, where both mechanisms may be active. Although the exact sequence of events is not clear, the global reaction rate for both mechanisms is first order with respect to CO at atmospheric pressure.

For the past few decades, significant amount of efforts has been put on CO oxidation and gas sensing. One-dimensional (1-D) semiconductor metal oxide nanostructures have attracted great attention due to their promising applications in nanodevices. Various 1-D metal oxide nanostructures, such as nanobelts, nanowires, nanoribbons, and nanofibers, have been used as transducers including ZnO, SnO₂, MoO₃, and Ga₂O₃ toward various analytes. These metal oxides have demonstrated high sensitivity, fast response/recovery time, and low cost, which arises from their unique combination of redox chemistry, optical, electrical and semiconductor properties. Such good gas sensing performances have played a key role in the successful implementation of chemical sensor technology for many years. Despite, the exciting results, which have been reported toward the gas sensing performance of metal oxides, the development of the ultra-sensitive, selective, and compact metal oxide based gas sensors for field still remains challenging. Nowadays, many efforts have been taken to enhance their sensitivity and selectivity, including heteroarchitectures, function with noble metal nanoparticles, and/or under the illumination of the lights.

Noble metal nanocrystals have been found to play important roles in a number of chemical reactions, which range from solar water splitting, CO oxidation, gas sensing to photodecomposition of organic pollutants. It is known that transition metal composite with metal oxide support can enhance the CO oxidation efficiency at the elevated temperature. There are also various studies of CO oxidation on noble metal/metal oxides catalysts, and several explanations of observation were given in the literature, including, for example: (1) a bifunctional mechanism based on the spillover of both CO and O₂ from the noble metal to the metal oxide, (2) a bifunctional mechanism but only restrict on CO, (3) the CO oxidation existence on a new phase, a Pt—Sn alloy, (4) the promoting of the CO oxidation on the adjacent SnO₂ due to the adsorption of the reactants on the Pt surface, and (5) the spillover of the O₂ from the SnO₂ to the Pt surface. However, each of the explanations mentioned above does not propose the way to minimize the CO poisoning effect and all experimental data was based on elevated temperatures.

### SUMMARY OF THE INVENTION

In consideration of the above issues, it would be desirable to have a system and method to obtain the optimum condition for Pt decorated SnO₂/SWNTs (Pt/SnO₂/SWNTs) and also correlate with its CO sensing performance. In accordance with an exemplary embodiment, light dependent sensing performance is disclosed with red, green and UV LED light under room temperature (or ambient temperature), and with the assistance of the UV LED light illumination, for example, the sensitivity of Pt/SnO₂/SWNTs can be enhanced to 2.1%/ppm_{V} to 50 ppm_{V} of CO and the detection limit can be down to 0.05 ppm_{V}.

In accordance with an exemplary embodiment, a method is disclosed of co-functionalizing single walled carbon nanotubes for sensing carbon monoxide at ambient temperature, the method comprising: electrochemically depositing a tin oxide (SnO₂) solution on aligned single-walled carbon nanotubes (SWNTs) to form SnO₂ functionalized SWNTs; electrochemically depositing a platinum (Pt) precursor solution on the SnO₂ functionalized SWNTs; and controlling applied potential and charge density during the electrochemical deposition of the platinum (Pt) solution on the SnO₂ functionalized SWNTs.

A gas sensor operable at ambient conditions is disclosed, the sensor comprising: co-functionalized platinum (Pt) and tin oxide (SnO₂) nanostructures on single-walled carbon nanotube (SWNTs) networks configured to detect carbon monoxide, and wherein the gas sensor has a sensitivity of at least 2.1%/ppm_{V} to 50 ppm_{V} of CO and a detection limit of at least 0.05 ppm_{V}.

It is to be understood that both the foregoing general description and the following detailed description are exemplary and explanatory and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIGS. 1A-1C are SEM images of SnO₂/SWNT before Pt deposition (FIG. 1A), Pt nanoparticles deposited on the same SnO₂/SWNT from 1 mM Pt precursor solution (deposition potential of −0.625 V and charge density of 0.125 C/cm²) (FIG. 1B), and Pt nanoparticles deposited on SnO₂/SWNTs from 10 mM Pt precursor solution (deposition potential at −0.625 V and charge density of 0.0125 C/cm²) (FIG. 1C), respectively.

FIGS. 2A-2B illustrate (a) resistance of SnO₂/SWNTs before and after the functionalization with Pt nanoparticles (FIG. 2A), and the I-V curves of SnO₂₁SWNTs, Pt/SnO₂/SWNTs(1), and Pt/SnO₂/SWNTs(10) (FIG. 2B), respectively, wherein the solid symbols represent the resistance of SnO₂/SWNTs, and open symbols represent the resistance after the Pt nanoparticles deposition with different precursor solution, respectively.

FIG. 3 illustrates electron transfer characteristics (Current (i_{SD}) vs. back gated voltage (V_{G})) of Pt decorated SnO₂/SWNTs hybrid nanostructures obtained from 1 and 10 mM Pt precursor solution and SnO₂/SWNTs, respectively.

FIGS. 4A-4B illustrate photocurrent sensing response under the illumination of red, green, and UV light for Pt/SnO₂/SWNTs hybrid nanostructures deposited from 1 mM (FIG. 4A) and 10 mM Pt solution (FIG. 4B), respectively.

FIGS. 5A-5B illustrate room temperature CO sensing of the Pt/SnO₂/SWNTs hybrid nanostructures deposited from 1 mM Pt precursor solution (FIG. 5A) and 10 mM Pt precursor solution (FIG. 5B), respectively, and wherein without illumination (black open square), under the illumination of red (red open circle), green (green open star) and UV light (purple open pentagon).

FIGS. 6A-6B illustrate room temperature UV illuminated CO sensing of the Pt/SnO₂/SWNTs hybrid nanostructures deposited from (a) 1 mM Pt precursor solution (FIG. 6A) and 10 mM Pt precursor solution (FIG. 6B).

FIGS. 7A-7B illustrate real time sensing performance of Pt/SnO₂/SWNTs (1 mM) towards CO of 50 to 200 ppm (FIG. 7A) and 5 to 200 ppm at room temperature (FIG. 7B).

FIGS. 8A-8B illustrate real time sensing performance of Pt/SnO₂/SWNTs (10 mM) towards CO of 50 ppm to 200 ppm (FIG. 8A) and 5 ppm to 200 ppm at room temperature (FIG. 8B).

FIG. 9A illustrates real time CO sensing performance at 100° C. for Pt/SnO₂/SWNTs hybrid nanostructures (from 1 mM Pt precursor solution and 10 mM Pt precursor solution, respectively) and FIG. 9B illustrates their calibration curve.

FIGS. 10A-10D illustrate NH₃ sensing in the absence of light and under the UV illumination of different hybrid nanostructures Pt/SWNTs (from 1 mM Pt; orange open square) (FIG. 10A), Pt/SnO₂/SWNTs (from 1 mM Pt; red open circle) (FIG. 10B), Pt/SnO₂/SWNTs (from 10 mM Pt; blue open triangle) (FIG. 10C), and the calibration curve for each type of hybrid nanostructures (FIG. 10D).

FIGS. 11A-11D illustrate NO₂ sensing in the absence of light and under the UV illumination of different hybrid nanostructures Pt/SWNTs (from 1 mM Pt; orange open square) (FIG. 11A), Pt/SnO₂/SWNTs (from 1 mM Pt; red open circle) (FIG. 11B), Pt/SnO₂/SWNTs (from 10 mM Pt; blue open triangle) (FIG. 11C), and the calibration curve for each type of hybrid nanostructures (FIG. 11D).

FIGS. 12A-12C are an exempalry sensor configuration, which includes sensor arrays with 15 microelectrodes as working electrodes (W.E.), integrated reference electrode (R.E.) and counter electrode (C.E.) (FIG. 12A), Teflon cell with test clips for SWNT alignment and functionalization (FIG. 12B); and sensing cell with gas inlet port and outlet port (12C).

### DETAILED DESCRIPTION

Reference will now be made in detail to the present preferred embodiments of the invention, examples of which are illustrated in the accompanying drawings. Wherever possible, the same reference numbers are used in the drawings and the description to refer to the same or like parts.

In accordance with an exemplary embodiment, Pt/SnO₂/SWNTs hybrid nanostructures were fabricated for CO sensing at room temperature. It should be mentioned that there are only a few CO sensors that can be operated at room temperature or near room temperature, but the sensitivity and selectivity is not competitive to the high temperature ones due to CO poisoning. In accordance with an exemplary embodiment, it is believed that the present disclosure demonstrates the first room temperature CO sensor that can detect CO down to 0.05 ppm_{V} at room temperature with the assistance of UV illumination.

### Experimental Procedure

### Electrode Micro Fabrication

In accordance with an exemplary embodiment, an individually addressable sensor array consisting of 15 microelectrodes with 3 micron gaps were microfabricated using our previously developed photolithography. A thermally oxidized highly doped p-type Si wafer (Ultrasil Corporation, Hayward, Calif.) with a 300 nm oxidation layer (grown by Low Pressure Chemical Vapor Deposition) was used as the substrate. First, a positive photoresist AZ 5214-E (AZ Electronics Materials USA Corporation, Somerville, N.J.) was spin-coated on the substrate at a speed of 1000 RPM for 2 seconds (s) followed by 3000 RPM for 30 second (s), and then the substrate was placed on a hot plate at 110° C. for 5 minutes (min). The patterns were photolithographically defined onto the substrates by UV light at a 70 mJ/cm² exposure dose and subsequently developed by immersing it in a developer (AZ 400K: DI water=1:4, AZ Electronics Materials USA Corporation, Somerville, N.J.) for 45 seconds. Afterwards, Ti (an adhesion layer) and Au films were e-beam deposited with film thicknesses of 20 nm and 180 nm, respectively. The substrate was soaked into an acetone bath overnight for the lift-off process. Prior to experiments, these prefabricated microelectrodes were diced, cleaned with acetone and water, and blown dry with ultra-high purity N₂ gas for further uses.

### SWNTs Alignment

In accordance with an exemplary embodiment, SWNTs were aligned through AC dielectrophoretic alignment. Carboxylated-SWNTs (SWNT-COOH 80-90% purity, Carbon Solution, Inc., Riverside, Calif., USA) were first dispersed (10 μg/mL) in N,N-dimethylforamide (DMF, Sigma Aldrich, MO, USA) and sonicated for 90 minutes at room temperature until a uniform suspension was obtained. The suspension was then transferred into a 50 mL Teflon centrifuge tube and then centrifuged for 90 minutes at 15,000 RPM at 23° C. (Beckman J2-HS and JA-17 rotor, Beckman Coulter, Inc., Brea, Calif., USA). After centrifuging, 10 mL of supernatant was removed immediately and put into a glass vial. The supernatant was then sonicated for an additional 60 minutes. Afterwards, 350 μL of the supernatant was placed into the customized Teflon cell with chip holder for SWNTs alignment. To obtain optimal SWNTs alignment, a 2 Vpp and 4 MHz frequency was applied to the electrodes for 10 seconds. A Labview program was designed to command the Keithley 3390 AC generator (aligner) and custom-made DAQ switcher for sequential SWNTs alignment. The synthesized sensors were rinsed with DI water, dried with Ultra High Purity (UHP) N₂ gas and then calcined at 300° C. under a reducing environment (forming gas, 5% of hydrogen in nitrogen) for 1 hour to remove the residue from the solution and to improve the connection.

### SWNTs Functionalization

### SnO₂ Functionalized SWNTs

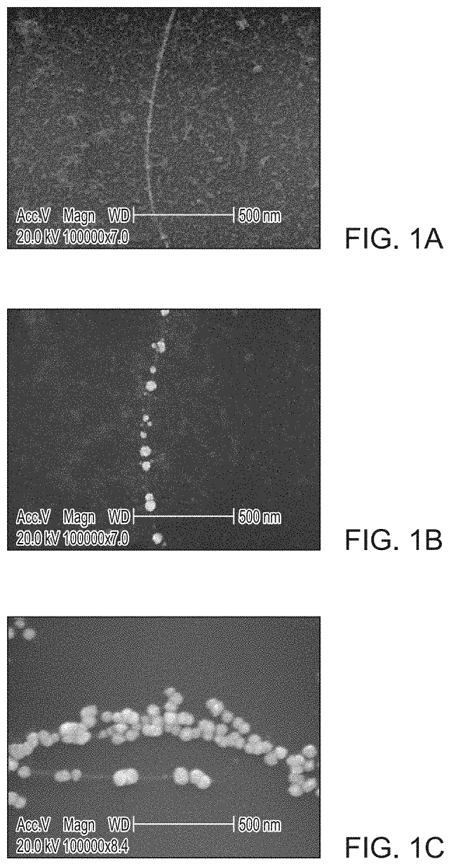

In accordance with an exemplary embodiment, the SnO₂/SWNTs hybrid nanostructures were obtained by functionalizing SWNTs with electrochemical deposition of tin dioxide from an electrolyte bath containing Sn²⁺. The electrolyte for electrochemical deposition was prepared according to the process as disclosed in Min, L.; Jae-Hong, L.; Syed, M.; Youngwoo, R.; Ashok, M.; Marc, A. D.; Nosang, V. M. Nanotechnology 2009, 20, 185602. 100 mM of sodium nitrate (≥99.0%, Sigma Aldrich, MO, USA) was added into 75 mM of nitric acid (70%, Sigma Aldrich, MO, USA) under constant stirring. 20 mM of tin chloride dehydrate (≥99.99%, Sigma Aldrich, MO, USA) was added with constant stirring for 12 hours prior to use. Finally, the pH of the solution was adjusted to 1.3 with hydrogen chloride (HCl). Linear Sweep Voltammetry (LSV) and chronoamperometry techniques were carried out at room temperature in the same customized Teflon cell with chip held (FIG. 1C) by using a three electrode electrochemical setup. A commercial potentiostat/galvanostat (SP-200, Potentiostat/Galvanostat/EIS, BioLogic Science Instruments, France) was used for electrochemical deposition, a Pt wire (99.99%, Sigma Aldrich, MO, USA) as the counter electrode and a saturated Ag/AgCl electrode (CHI-111, CH Instrument, Inc., Austin, Tex., USA) as the reference electrode. For example, an optimization process of the SnO₂ functionalized SWNTs hybrid can be obtained, for example, as disclosed in Heng C. Su, Miluo Zhang, Wayne Bosze, and Nosang V. Myung, Tin Dioxide Functionalized Single-Walled Carbon Nanotube (SnO₂/SWNT)—Based Ammonia Gas Sensors and Their Sensing Mechanism, Journal of the Electrochemical Society 2014 161: B283-B290, which is incorporated herein in its entirety.

### Pt Nanoparticles Decorated SnO₂/SWNTs

The Pt decorated SnO₂/SWNTs hybrid nanostructures were obtained from two different concentrations of Pt electrodeposition precursor solution, 1 mM and 10 mM K₂PtCl₄ solution, respectively, with 10 mM of KCl as the supporting electrolyte. LSV was utilized to characterize the electrochemical deposition of Pt. The ability to tune Pt particle size, particle density, and its morphology was demonstrated by controlling the applied potential and charge density. The deposition potential and charge density were optimized for CO sensing purpose.

### Electrical and Sensing Property Measurements and Material Characterization

The morphologies and compositions of the Pt/SnO₂/SWNTs hybrid nanostructures were investigated using field emission-scanning electron microscopy (FE-SEM, FEG-XL30, Philips) and energy-dispersive X-ray spectroscopy (EDS). The electrical resistance and FET properties of these hybrid nanostructures were determined by two-probe measurements with a Keithley 236 source.

### Gas Sensing in the Absence of the Light

In accordance with an exemplary embodiment, the sensing performance of the Pt/SnO₂/SWNTs nanostructures was studied by installing the sensing chips in a customized sensing cell (maximum three chips in three separate cells at one time) with inlet and outlet ports for gas flow and then clipping the chips to a Keithley 236 source to obtain an electrical connection. The electrical resistance was continuously recorded under an applied voltage fixed at 1 V. During the sensing measurement, the total gas flow rate was fixed at 200 sccm (standard cubic centimeters per minute) with desired analyte diluted with dry air. In all sensing experiments, sensors were first stabilized in dry air for 60 minutes, and then challenged with different concentrations of analyte with 15 minutes exposure and 20 minutes recovery times. The sensor response was determined by the resistance change before and after exposure to analyte and defined as (R_{f}-Rₒ)/Rₒ, where R_{f} is the final resistance of the peak height and Rₒ is initial baseline resistance prior to analyte exposure. In accordance with an exemplary embodiment, the response time is defined as the time for the sensor to reach 90% of its steady-state value, and the recovery time is identified as the time required for the sensor after the exposure to return to 50% of its maximum response.

In accordance with an exemplary embodiment, the sensing performance at 100° C. was carried out to understand the effect of the environment temperature and the relief of the CO poisoning effect. Before gas sensing, the entire sensing unit including Teflon cell and clip system was put in the oven (DVS 402, Yamato) for 30 minutes to stabilize the temperature. The sensing process was the same as mentioned above.

### Light Dependent Gas Sensing Performance

In accordance with an exemplary embodiment, in order to further investigate the light dependent property of the Pt/SnO₂/SWNTs nanostructures, red (λ=630 nm, 7.2 mW, photon flux F=1.44×10¹⁴/cm²s, LED630E), green (λ=525 nm. 2.6 mW, photon flux F=4.34×10¹³/cm²s, LED525E), and UV (λ=370 nm, 2.5 mW, photon flux F=2.98×10¹³/cm²s, LED370E) LED lights (Thorlabs, N.J., USA) were integrated into the customized sensing cell. The photocurrent was carried out at the room temperature with the Keithley 236 source meter continuously recorded under an applied voltage fixed at 1 V. For each LED light photocurrent measurement, the light was on for 15 minutes exposure and off for 20 minutes recovery times for three cycles under the flow of dry air at 200 sccm. While gas sensing under different LED light illumination, LED lights were continuous on for entire sensing process.

### Fabrication, Optimization and Characterization of Pt/SnO₂/SWNTs

In accordance with previous work, the optimization of SnO₂/SWNTs toward CO sensing have been studied. Here, in accordance with an exemplary embodiment, electrodeposited Pt nanoparticles on SnO₂/SWNTs were studied for better CO sensing performance at room temperature and less CO poisoning. FIGS. 1A and 1B show the SnO₂/SWNTs hybrid nanostructures before and after the Pt electrodeposition using 1 mM K₂PtCl₄ solution, respectively. FIG. 1C shows the hybrid nanostructures after electrodeposition of Pt nanoparticles from 10 mM K₂PtCl₄ solution. The applied potential was fixed at −0.625 V versus sat. Ag/AgCl. Pt particle size and density of the three configurations (Pt/SWNTs, Pt/SnO₂/SWNTs obtained from 1 mM K₂PtCl₄ solution, and Pt/SnO₂/SWNTs fabricated from 10 mM H₂PtCl₆ solution) were compared in Table 1 for better understanding. Compared to direct electrodeposition of Pt nanoparticles on SWNTs, Pt nanoparticles on SnO₂/SWNTs were larger with lower particle density per SWNTs, which might be attributed to higher surface overpotential. Once the nuclei are formed, the grains grow rapidly, resulting in the formation of large Pt nanoparticles.

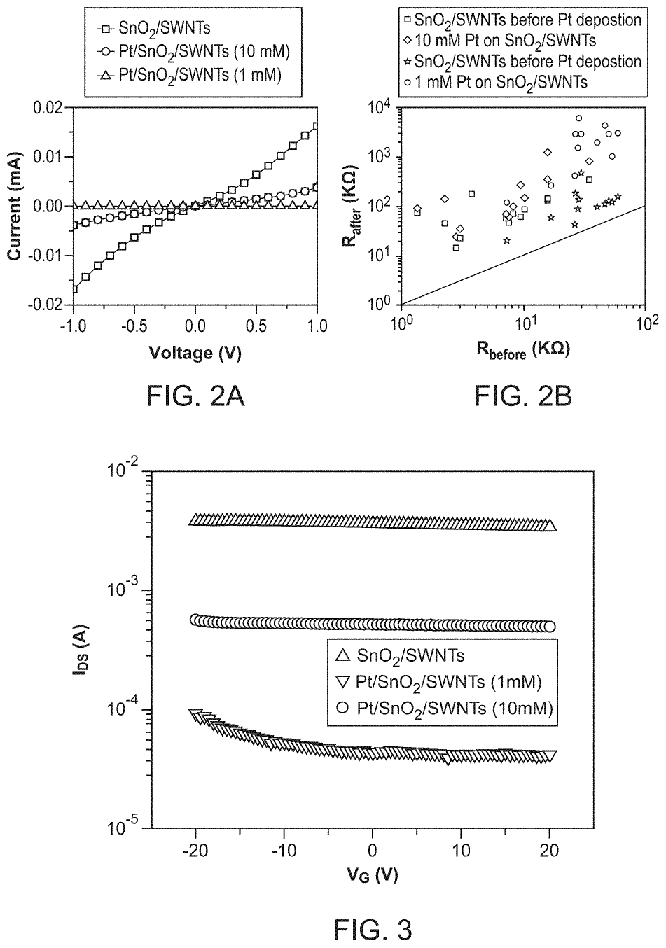

In accordance with an exemplary embodiment, I-V curve in FIG. 2A reveals the electrical conductivity change, which is consistent with the SEM images taken from the same sensor before and after Pt nanoparticle deposition. The electrical resistances change before and after Pt deposition is listed in FIG. 2B. As shown, Pt nanoparticles deposited on SnO₂/SWNTs work as the scattering sites of the carriers, leading to the decrease of the electrical conductivity. However, the electrical conductivity of Pt/SnO₂/SWNTs from 10 mM solution is slightly higher than that of Pt/SnO₂/SWNTs from 1 mM solution, which can be due to the growth of the particle density and the particle size, which leads to the interconnection of Pt nanoparticles and creates another carrier pathway; thus enhance the conductivity slightly. In accordance with an exemplary embodiment, the electron transfer characteristics are measured in ambient condition and plotted in FIG. 3. Both Pt/SnO₂/SWNTs(1 mM) and Pt/SnO₂/SWNTs(10 mM) show p-type semiconductor behavior as expected.

TABLE 1
The particle size and density of Pt/SWNTs and Pt decorated
SnO₂/SWNTs, and wherein the deposition potential and charge
density were fixed at −0.625 V and 0.0125 C/cm², respectively.

| | | Pt/SnO₂/SWNTs | Pt/SnO₂/SWNTs |
|---|---|---|---|
| | Pt/SWNTs | (1 mM) | (10 mM) |
| Particle size (nm) | 34.8 ± 3.2 | 82.9 ± 12.1 | 84.6 ± 2.8 |
| Particle density | 70.7 ± 3.8 | 20 ± 3.0 | 52.3 ± 2.1 |
| (number/per | | | |
| SWNT) | | | |

### Sensing Performance of Pt/SnO₂/SWNTs in the Absence of Light

### CO Sensing Performance at Room Temperature (RT)

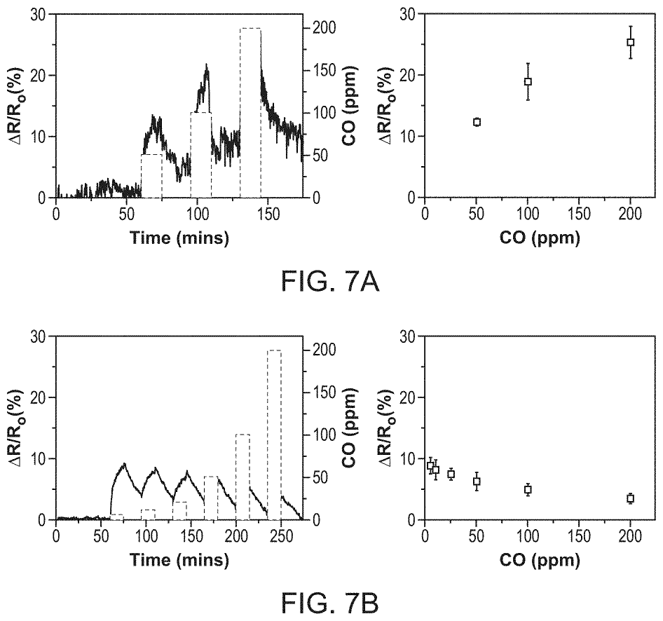

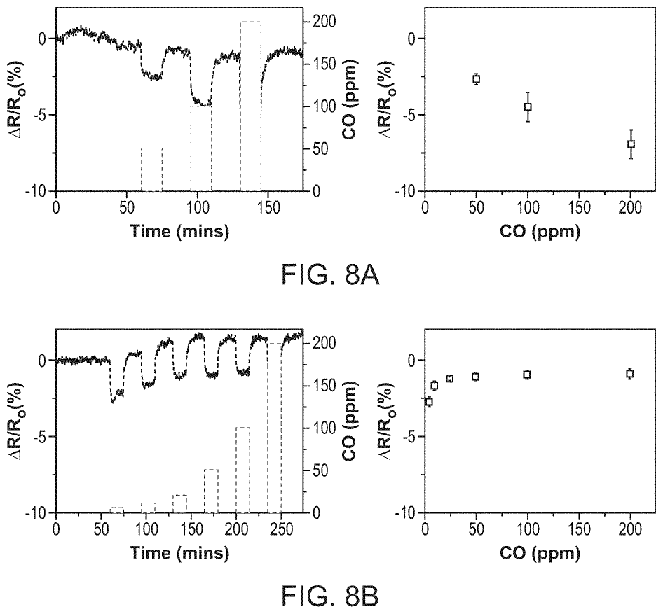

In accordance with an exemplary embodiment, in order to understand the sensing performance of Pt/SnO₂/SWNTs, a preliminary CO sensing was carried out for the concentration of 50, 100, and 200 ppm_{V}, FIG. 7A for Pt/SnO₂/SWNTs(1 mM) and FIG. 8A for Pt/SnO₂/SWNTs(10 mM), respectively. After that, their whole sensing performance was examined from 5 to 200 ppm_{V} of CO, and corresponding data are shown in FIG. 7B and FIG. 8B, respectively. Comparing the preliminary and full concentration sensing results of Pt/SnO₂/SWNTs(1 mM), the sensing responses towards both three-concentration (FIG. 7A) and six-concentration of CO (FIG. 8B) were improved for Pt/SnO₂/SWNTs(1 mM) compared to SnO₂/SWNTs. For example, the resistance increased around 21% when exposed to 200 ppm_{V} CO (FIG. 7A), compared to that of 15% for Pt/SWNTs (data not shown). In accordance with an exemplary embodiment, for Pt/SnO₂/SWNTs(10 mM), the sensing performance was also improved, especially the response and recovery time was quite reduced, FIG. 8B. The opposite sensing pattern of the Pt/SnO₂/SWNTs(1 mM) and Pt/SnO₂/SWNTs(10 mM) indicates the typical p-type and n-type semiconductor sensing behavior for Pt/SnO₂/SWNTs(1 mM) and Pt/SnO₂/SWNTs(10 mM), respectively. In accordance with an exemplary embodiment, CO poisoning was observed for both sensors, FIG. 7B and FIG. 8B, where the sensing performance is negatively proportional to the increase of the CO concentration. Details are summarized in Table 2.

TABLE 2
Comparison of room temperature CO sensing properties of
Pt/SWNTs and Pt/SnO₂/SWNTs hybrid nanostructures
fabricated under optimum condition, respectively.

| | | Pt/SnO₂/SWNTs | Pt/SnO₂/SWNTs |
|---|---|---|---|
| | Pt/SWNTs | (1 mM) | (10 mM) |
| CO sensitivity | 0.05 | 0.14 | 0.04 |
| (%/ppmᵥ) | | | |
| Response time t₉₀ | 9.2 | 8.4 | 3.7 |
| at 50 ppm (min) | | | |
| Recovery time t₅₀ | 16.2 | 13.5 | 5.8 |
| at 50 ppm (min) | | | |

### CO Sensing Mechanism of Pt/SnO₂/SWNTs

Various researches have studied the CO oxidation on either Pd/SnO₂ or Pt/SnO₂ catalysts but there are very limited reports on using this combination of Pt/SnO₂/SWNTs as CO gas sensor at room temperature. As mentioned in the introduction section, there are five different hypothesizes or assumptions based on the observation of the CO behavior on catalysts. Here, due to two different CO sensing patterns, the sensing mechanism of Pt/SnO₂/SWNTs(1 mM) and Pt/SnO₂/SWNTs(10 mM) will be explained separately.

CO Sensing Mechanism on Pt/SnO₂/SWNTs from the 1 mM Pt Precursor Solution (Pt/SnO₂/SWNTs(1 mM))

In accordance with an exemplary embodiment, the CO sensing performance of Pt/SnO₂/SWNTs(1 mM) can be explained that CO mainly adsorbs on the Pt surface and only very limited amount CO adsorbs on SnO₂ surface, followed by the migration of CO to the junction of Pt and SnO₂/SWNTs via surface diffusion. While the SnO₂ surface adsorbs O₂, O₂⁻ and O⁻, which diffuse to Pt and react with adsorbed CO via L-H step releasing CO₂ as product. In accordance with an exemplary embodiment, CO oxidation also exists on Pt surface as described in the section of Pt/SWNTs hybrid nanostructures. However, owing to the higher sticking coefficient of CO than O₂, CO obstructs the O₂ adsorption on Pt, which consequently inhibits CO oxidation and causes CO poisoning.

After reaction, the electrons are released back into the hybrid nanostructures, leading to the recombination of electrons and electrons holes, which can result in the decreasing of the electrical conductivity. Thus, the electrical resistance increases during CO sensing progress. In accordance with an exemplary embodiment, the hypothesis mentioned above have been verified by the simulation via Monte Carlo methods with 4 wt. % Pt loading on SnO₂. In the simulation, with solely Pt and or Pt/SnO₂, the oxidation process is negatively proportional to the CO concentration in line with our sensing performance.

CO Sensing Mechanism on Pt/SnO₂/SWNTs from the 10 mM Pt Precursor Solution (Pt/SnO₂/SWNTs(10 mM))

Although the electron transfer characteristics of Pt/SnO₂/SWNTs(10 mM) show a weak p-type semiconducting behavior (FIG. 3), the CO sensing performance in FIGS. 8A and 8B indicates a n-type semiconducting sensing mechanism. In accordance with an exemplary embodiment, this might be due to the increase of the Pt particle density resulting in the larger surface active sites. Since CO mainly adsorbs on the Pt surface, the increase of particle density (from 20 to 50 particles/per SWNT) with similar particle size, generates more surface area for CO oxidation, thus improves the response time, FIGS. 8A and 8B. In accordance with an exemplary embodiment, the increase of the particle density also creates the chance for the Pt cluster to connect with each other and provides another electrons pathway. After CO oxidation, the released electron flow into the Pt clusters instead of flowing back to the hybrid nanostructures causing the separation of the electrons and electron holes. Consequently, the separated electron holes move back to SWNTs and increase the conductivity.

### CO Sensing at Elevated Temperature

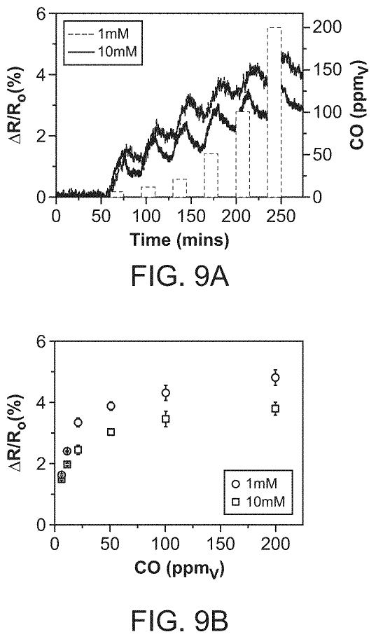

In accordance with an exemplary embodiment, CO oxidation efficiency can be promoted at increased temperature which reaches maximum efficiency around 170° C. In order to improve CO sensing performance and minimize CO poisoning effect, the hybrid nanostructures were tested at the elevated temperature. FIGS. 9A and 9B reveal the real time CO sensing performance at 100° C. for Pt/SnO₂/SWNTs hybrid nanostructures. For the circumstance of Pt/SnO₂/SWNTs (1 mM), although the response decreases (for example, 3% compared to 13% at room temperature for 50 ppm_{V} CO), CO poisoning is suppressed. In accordance with an exemplary embodiment, this result is owing to CO desorption rate enhancement and CO migration on Pt surface. In accordance with an exemplary embodiment, it is believed that the adsorption of the CO is strongly favored at room temperature but gradually lose as temperature increase. In another words, the CO surface coverage is inversely proportional to the temperature, consistent with what another metal/SnO₂ hybrid nanoparticles sensing toward CO at 100° C.

In accordance with an exemplary embodiment, in the case of the Pt/SnO₂/SWNTs(10 mM), the CO poisoning was also reduced and it showed a p-type semiconducting behavior opposite to the sensing pattern at room temperature. In accordance with an exemplary embodiment, this might be attributed to the change of surface chemistry at elevated temperature. Pt/SWNTs were also tested at the elevated temperature, but the sensing performance is not enhanced as much as Pt/SnO₂/SWNTs (data not shown here).

### Sensing Performance of Pt/SnO₂/SWNTs Under the Illumination of the LED Lights

### Photocurrent Measurement

Although sensing at the elevated temperature can effectively reduce CO poisoning, the sensing response undesired decreases significantly. In order to minimize CO poisoning while maintain or enhance the CO sensing response, the sensing with assistance of different wavelengths of lights was systematically investigated.

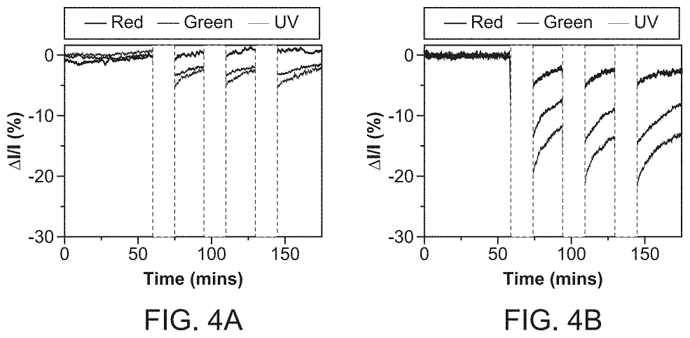

Photoelectrical measurements of the Pt/SnO₂/SWNTs hybrid nanostructures were carried out with red (630 nm, 7.2 mW, photon flux F=1.44×10¹⁴/cm²s), green (525 nm. 2.6 mW, photon flux F=4.34×10¹³/cm²s), and UV (370 nm, 2.5 mW, photon flux F=2.98×10¹³/cm²s) LED lights, respectively. FIGS. 4A and 4B show the photocurrent sensing response of Pt/SnO₂/SWNTs hybrid nanostructures under different wavelengths of lights. The Pt/SnO₂/SWNTs first experience the reduction of the current upon the illumination of light (where ΔI/Iₒ is defined as the difference between the illuminated and dark current values divided by the dark current), and then slowly recovered after the light turn off. FIG. 4A reveals the typical Pt/SnO₂/SWNTs photocurrent response, where two processes have occurred in the metal oxide upon LED light exposure. Firstly, photons (such as UV photons) whose energy exceed the band gap energy of SnO₂ (E_{g}=3.6 eV) can photogenerate electron-hole pairs (Equation 6). Secondly, the surface adsorbed oxygen molecules are photodesorbed upon the exposure of light (Equation 7). After the light is off, the desorbed oxygen molecules will re-adsorb onto the SnO₂ surface and thus withdraw the electrons from the hybrid nanostructures, resulting in the increase of the conductivity (Equation 8).

hv→e⁻+h⁺ (Eq. 6)

O₂⁻(ad)+h⁺O₂(g) (Eq. 7)

O₂(g)+e⁻→O₂⁻(ad) (Eq. 8)

In accordance with an exemplary embodiment, in the case of Pt/SnO₂/SWNTs(1 mM), under the red (λ=630 nm˜2 eV) light, the photon energy is much lower than the band gap energy of SnO₂ (3.6 eV); therefore, there is no photocurrent change. For green light (λ=525 nm˜2.36 eV) and UV light (λ=370 nm˜3.35 eV), the photon energy is higher than the bad gap energy of SnO₂, which excites electron-hole pairs and induce a ca. 5% decrease of the current.

In accordance with an exemplary embodiment, in the case of Pt/SnO₂/SWNTs(10 mM), there was a 5% change at red light illumination and more significant change under green (15%) and UV light (22%). This might be due to the effect of the localized surface plasmon resonance (LSPR). Two possible mechanisms plasmonic enhancement of the light adsorption and plasmonic sensitization are involved. Upon irradiation of the light, the incident light excites the LSPR in the noble metal nanocrystals and generates electron-hole pairs in the semiconductor simultaneously. The part of the semiconductor that is adjacent to the metal nanoparticles is equivalent to be put into a strong local electric field whose intensity is several order magnitude higher than the incident light. In accordance with an exemplary embodiment, due to the high density of Pt nanoparticles in Pt/SnO₂/SWNTs(10 mM), this electric field is further enhanced. Therefore, in the enhanced electric field, the generation rate of the electron-hole pairs can be greater promoted. In addition, under the resonant excitation of the metal nanoparticles, a population of electrons are generated and no longer in their thermal equilibrium. Those hot electrons (energetic electrons) with energy higher than the Schottky barriers can cross barriers and inject into the conduction band of the SnO₂. The excess electrons then flow into the SWNTs and recombine the electron holes, resulting in the decrease of the conductivity.

### CO Sensing Under the Illumination of LED Light and Sensing Mechanisms

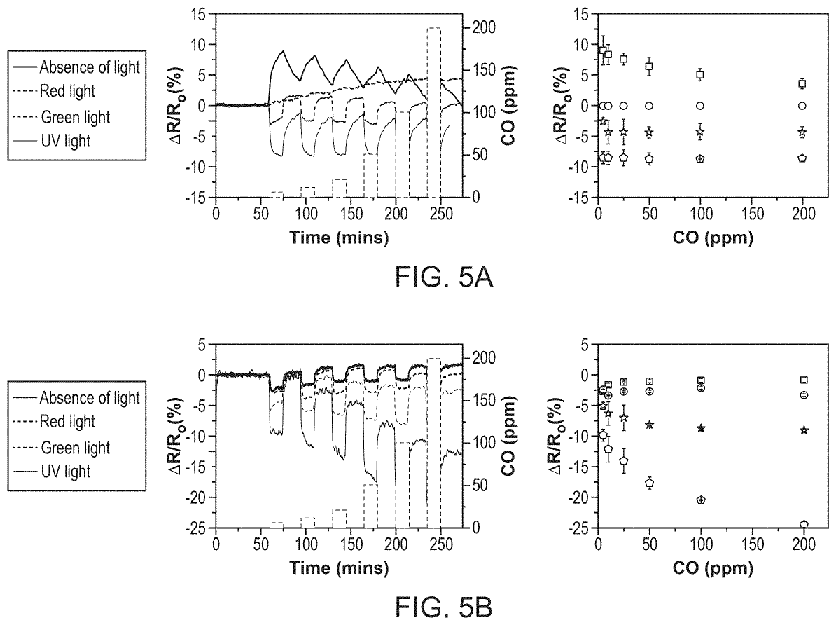

FIGS. 5A and 5B show the CO sensing under the illumination of red, green and UV light from 5 ppm_{V} to 200 ppm_{V} in room temperature for the Pt/SnO₂/SWNTs hybrid nanostructures. FIG. 5A displays the CO sensing performance of Pt/SnO₂/SWNTs(1 mM) in the absence of light, under red, green, and UV light, respectively. In accordance with an exemplary embodiment, first, under the radiance of red light, the adsorption energy for CO on Pt (126-176 kJ/mol) falls in the range of the red light. Therefore, CO adsorption is inhibited and results in the continuous increase of the electrical resistance. Second, under the illumination of the green and UV light, the Pt nanoparticles work as the amplifiers for the incident light to enhance the generation of the electron-hole pairs. The electrons were captured by CO molecules to form some surface adsorbed molecules, which continue to oxide CO by L-H mechanism. At the same time, the electron holes flow into the SWNTs, which increase the hybrid nanostructures conductivity. Under this circumstance, the conductivity of entire hybrid nanostructures increases upon CO exposure. In accordance with an exemplary embodiment, the last point is that the dynamic range of CO sensing is narrow under the assistance of the light illumination.

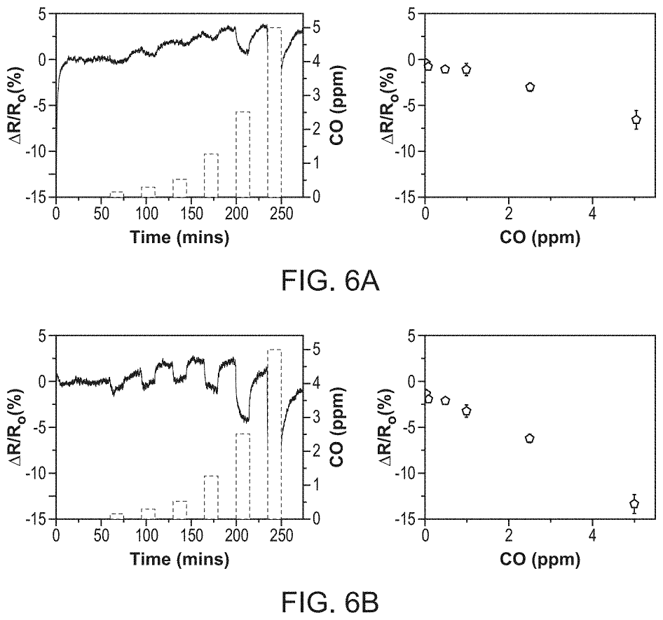

The CO sensing mechanism of Pt/SnO₂/SWNTs(10 mM) under light illumination is the same with that of Pt/SnO₂/SWNTs(1 mM) nanostructures. The sensing performance and dynamic range can be further improved (FIG. 5B) because the higher density of the Pt nanoparticles enhance the generation of electron-hole pairs. FIGS. 6A and 6B show that with the assistance of UV light, the detection limit of these Pt/SnO₂/SWNTs(10 mM) hybrid nanostructures towards CO is down to 0.05 ppm_{V}, which is the lowest for room temperature CO sensors so far.

NH₃ and NO₂ Sensing Under the Illumination of LED Light

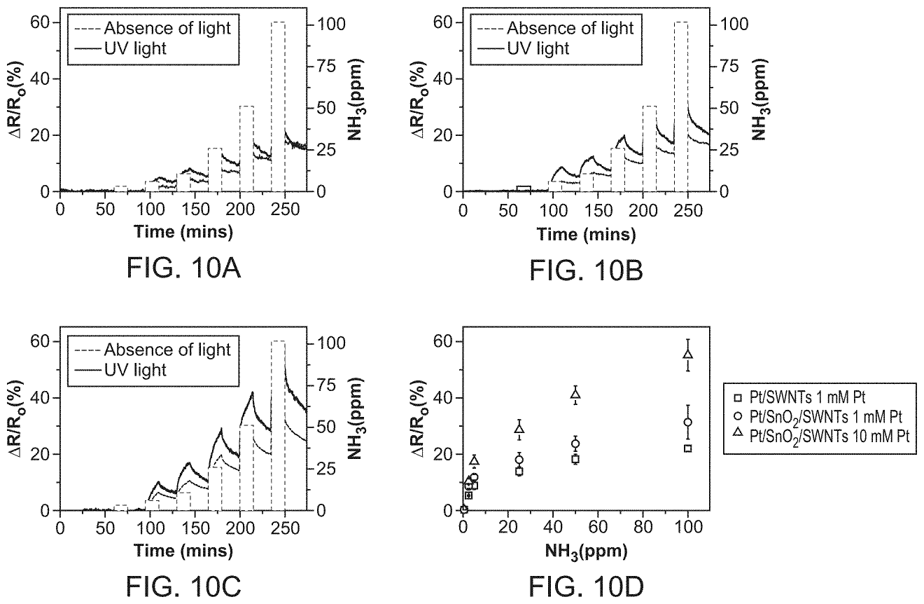

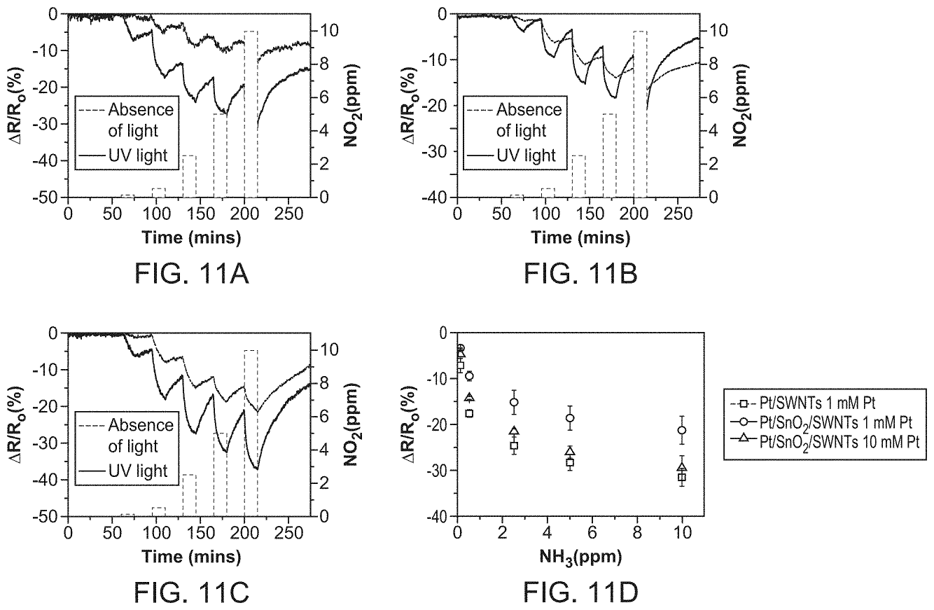

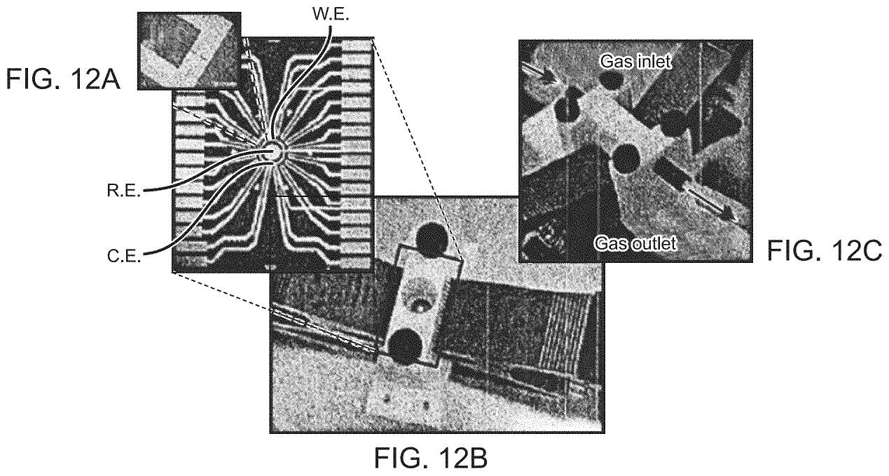

In accordance with an exemplary embodiment, the sensors were also challenged with background analytes such as NH₃ and NO₂ to identify the potential interference and demonstrate the sensor viability in the practical application. FIGS. 10A-10D and FIGS. 11A-11D show the sensing performance of Pt/SWNTs and Pt/SnO₂/SWNTs with and without the light illumination. In general, the sensitivity, response, and recovery time were promoted under light illumination. Morphology-dependent sensing performance was observed for Pt/SnO₂/SWNTs especially for the case of the NH₃ (FIGS. 10A-10D), the response doubled compared to the one sensing in the absence of the light, which is consistent with previously published results. Overall, the existing of the Pt nanoparticles boosts the NH₃ adsorption and then enhances the sensing performance. In accordance with an exemplary embodiment, this phenomena could be amplified under the illumination of the UV light owing to generation of the extra electron-hole pairs speeding up surface reaction for NH₃.

In accordance with an exemplary embodiment, for the case of the NO₂ sensing under the UV light illumination (FIGS. 11A-11D), the response and recovery time were improved. This is because that the electron holes from photo-generated electron-hole pairs recombine with the NO₂ and will be adsorbed on the surface under the UV light.

In accordance with an exemplary embodiment, SWNTs based hetero-nanostructures room temperature CO gas sensors are disclosed, and wherein a sequential electrochemical deposition approach was used to functionalize SWNTs with SnO₂ followed by Pt nanoparticles electrodeposition. In accordance with an exemplary embodiment, shape, morphology, and size of the Pt particles were controlled by adjusting the electrodeposition conditions and electrolyte composition. In accordance with an exemplary embodiment, the Pt nanoparticles density in Pt/SnO₂/SWNTs hybrid nanostructures are controlled by Pt precursor solution concentration (for example, 1 mM Pt and 10 mM Pt precursor solution from K₂PtCl₄).

Sensing results indicate the enhancement of the sensitivity toward CO by Pt functionalized SWNTs, especially Pt/SnO₂/SWNTs hybrid nanostructures. The enhancement in the sensing performance observed for Pt/SnO₂/SWNTs hybrid nanostructures is attributed to the Pt nanoparticles, which serve as catalytic activators for CO oxidation. Additionally, with the assistance of UV illumination, the CO poisoning was eliminated and the room temperature limit of detection is as low as 0.05 ppm_{V} (Pt/SnO₂/SWTNs(10)). The sensors are also tested against background analytes to demonstrate viability in the field.

It will be apparent to those skilled in the art that various modifications and variation can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A method of co-functionalizing single walled carbon nanotubes for sensing carbon monoxide at ambient temperature, the method comprising:
electrochemically depositing a tin oxide (SnO₂) solution on aligned single-walled carbon nanotubes (SWNTs) to form SnO₂ functionalized SWNTs;
electrochemically depositing a platinum (Pt) precursor solution on the SnO₂ functionalized SWNTs; and
controlling applied potential and charge density during the electrochemical deposition of the platinum (Pt) solution on the SnO₂ functionalized SWNTs.

2. The method of claim 1, wherein the platinum (Pt) precursor solution is a K₂PtCl₄ solution.

3. The method of claim 1, wherein the platinum (Pt) precursor solution is a solution with between 1 mM and 10 mM K₂PtCl₄ solution.

4. The method of claim 3, wherein the K₂PtCl₄ solution has 10 mM of KCl as a supporting electrolyte.

5. The method of claim 1, comprising:
sensing carbon monoxide at ambient temperature with platinum and tin oxide co-functionalized single walled carbon nanotubes (Pt/SnO₂/SWNTs).

6. The method of claim 1, comprising:
assembling the platinum and tin oxide co-functionalized single walled carbon nanotubes (Pt/SnO₂/SWNTs) on microelectrodes for carbon monoxide sensing.

7. The method of claim 1, comprising:
functionalizing the platinum and tin oxide co-functionalized single walled carbon nanotubes (Pt/SnO₂/SWNTs) to have a sensitivity of at least 2.1%/ppm_{V} to 50 ppm_{V} of CO and a detection limit of at least 0.05 ppm_{V}.

8. The method of claim 1, comprising:
setting a deposition potential at −0.625 V and a charge density of 0.0125 C/cm² during the electrochemical deposition of the platinum (Pt) solution onto the SnO₂ functionalized SWNTs.

9. The method of claim 1, comprising:
preparing a carbon nanotube suspension of carboxylated single-walled carbon nanotubes in a solution of N, N-dimethylformamide;
sonicating the solution until a uniform suspension is obtained;
centrifuging the suspension and collecting a supernatant;
placing the supernatant into a Teflon cell with a chip for SWNT alignment; and
obtaining alignment of the single-walled carbon nanotubes (SWNTs) across the microelectrodes.

10. The method of claim 9, comprising:
obtaining alignment by applying a 2 peak to peak voltage (Vₚₚ) and a 4 MHz frequency.

11. A gas sensor operable at ambient conditions, the sensor comprising:
co-functionalized platinum (Pt) and tin oxide (SnO₂) nanostructures on single-walled carbon nanotube (SWNTs) networks configured to detect carbon monoxide, and wherein the gas sensor has a sensitivity of at least 2.1%/ppm_{V} to 50 ppm_{V} of CO and a detection limit of at least 0.05 ppm_{V}.

12. The sensor of claim 11, further comprising:
a substrate configured to receive the co-functionalized platinum and tin oxide nanostructures on single-walled carbon nanotube (SWNTs) networks;
a plurality of working electrodes; and
a sensing cell having a gas inlet and a gas outlet.

13. The sensor of claim 11, wherein the co-functionalized platinum and tin oxide nanostructures on single-walled carbon nanotube (SWNTs) networks are formed by a process comprising:
electrochemically depositing a tin oxide (SnO₂) solution on aligned single-walled carbon nanotubes (SWNTs) to form SnO₂ functionalized SWNTs;
electrochemically depositing a platinum (Pt) precursor solution on the SnO₂ functionalized SWNTs; and
controlling applied potential and charge density during the electrochemical deposition of the platinum (Pt) solution on the SnO₂ functionalized SWNTs.

14. The sensor of claim 13, wherein the platinum (Pt) precursor solution is a K₂PtCl₄ solution.

15. The sensor of claim 14, wherein the platinum (Pt) precursor solution is a solution with between 1 mM and 10 mM K₂PtCl₄ solution, with 10 mM of KCl as a supporting electrolyte.

16. The sensor of claim 15, comprising:
setting a deposition potential at −0.625 V and a charge density of 0.0125 C/cm² during the electrochemical deposition of the platinum (Pt) solution onto the SnO₂ functionalized SWNTs.

17. The sensor of claim 11, further comprising:
an ultraviolet (UV) light source configured to improve sensitivity of the gas sensor.

18. The sensor of claim 17, wherein the UV light source is an LED light.
